(19) United States
(12) Patent Application Publication
(10) Pub. No.: US 2004/0162577 A1
(43) Pub. Date: Aug. 19, 2004

(54) Shielded pressure placement device and method

(76) Inventor: Theodore T. Tarone, West Palm Beach, FL (US)

Correspondence Address: Matthew W. Siegal, Stroock & Strook & Lavan LLP, United States, 180 Maiden Lane, New York, NY, 10038, US

(21) Appl. No.: 10/693,445

(22) Filed: Oct. 24, 2003

Related U.S. Application Data
(60) Provisional application No. 60/421,005, filed on Oct. 24, 2002.

(51) Int. Cl. A61B 17/00

(52) U.S. Cl. 606/201

(57) ABSTRACT

A shielded pressure placement device for the reduction of bleeding or edema and exposure to radiation is disclosed, including a flexible medium for receiving an application of pressure, a radiation shield operatively engaged to the flexible medium, and wherein the radiation shield transmits the application of pressure to a patient to reduce bleeding or edema and wherein the radiation shield reduces radiation exposure to a user.

### CROSS-REFERENCE TO RELATED APPLICATIONS

This application claims the benefit of U.S. Provisional Application Serial No. 60/421,005, filed on Oct. 24, 2002, entitled SHIELDED PRESSURE PLACEMENT. DEVICE AND METHOD.

### TECHNICAL FIELD

The present invention relates to radioactive implants for medical therapeutic purposes, referred to in the art as “radioactive seeds,” “seeds,” or “sources” for therapeutic radiation treatment of oncological and other medical conditions. More particularly, the present invention is directed to a novel shielded pressure placement device for the reduction of bleeding or edema and exposure to radiation in interstitial implantation brachytherapy and also for general brachytherapy treatments.

### BACKGROUND OF THE INVENTION

Brachytherapy is a form of cancer treatment in which a radioactive energy source, such as an isotope “seed,” is placed into or adjacent to a malignant tumor. In order to place isotope seeds into proper position, needles, using a two-dimensional grid pattern in conjunction with longitudinal spacing, are typically used to deliver isotope seeds. The two dimensional grid is frequently defined by a needle guide, called a template. The template is provided with a plurality of holes that provide guidance for the longitudinal progression of the needles, thus insuring their desired two-dimensional position within the tumor. After the two-dimensional needle array is positioned within the tumor, the isotope seeds are deposited along the longitudinal axis of each needle. The repeated insertion of the needle used in the brachytherapy procedure can cause bleeding or edema.

Edematous conditions, i.e., excessive accumulation of fluid in tissues, are painful conditions that can arise from a variety of causes. For example, operative and postoperative conditions can cause blood stasis and venous thromboembolism, a serious edematous condition. The swelling in edematous conditions can be unsightly and ultimately life threatening.

It is well known to treat edema with pressure devices that squeeze limbs, typically by means of an inflatable pressure cuff wrapped around the limb. The pressure device moves excess fluid from engorged tissues from distal portions of the limb to proximal portions, eventually to the trunk of the body where the fluids are absorbed in the circulatory system and excreted from the body. These pressure devices thus perform external, non-invasive compression therapy.

However, the prior art does not disclose or teach a device that can be used to perform external compression therapy to reduce bleeding or edema in a patient while reducing a medical professional's exposure to radiation from implanted radioactive energy sources. Thus, a need exists for a post-implantation pressure placement device that includes a radioactive shielding element.

### SUMMARY OF THE INVENTION

The present invention eliminates the above-mentioned needs by providing a post-implantation pressure placement device that includes a radioactive shielding element. The device is designed to permit the application of pressure to the perineum of a patient after repeated insertions of needles into the patient during a typical interstitial implantation brachytherapy and general brachytherapy treatments where radioactive seeds are implanted in the patient.

In accordance with the present invention, there is provided a shielded pressure placement device for the reduction of bleeding or edema and exposure to radiation, including a flexible medium for receiving an application of pressure, a radiation shield operatively engaged to the flexible medium, and wherein the radiation shield transmits the application of pressure to a patient to reduce bleeding or edema and wherein the radiation shield reduces radiation exposure to a user.

The present invention is further directed to a method for applying pressure to a surgical patient, the method including the steps of applying pressure to a flexible medium, the flexible medium transmitting the pressure to a radiation shield, transmitting the pressure from the radiation shield to a body region of a surgical patient, and reducing radiation exposure to a user by the radiation shield.

### BRIEF DESCRIPTION OF THE DRAWINGS

FIG. 1 is a side view illustration of the preferred embodiment of the present invention.

FIG. 2 is a side view illustration an alternative embodiment of the present invention of FIG. 1.

### DETAILED DESCRIPTION OF THE PREFERRED EMBODIMENT

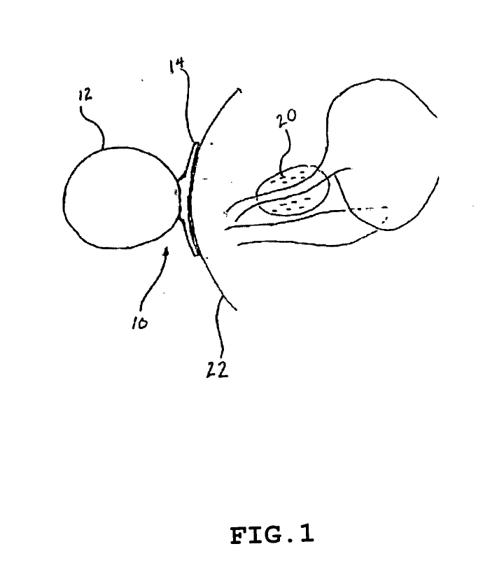

Referring now to FIG. 1, the preferred embodiment of the present invention is illustrated as pressure placement device 10. Pressure placement device 10 includes a flexible medium 12 for receiving an application of pressure. A user, such as a medical professional, applies the pressure directly to flexible medium 12. Flexible medium 12 can be formed from any one of numerous flexible materials know in the art, such as rubbers, polymers, gels, foams, and the like. If flexible medium 12 is a gel or foam, for example, various materials (not shown) may be used to cover or encapsulate flexible medium 12 in order to better contain flexible material 12. Preferably, flexible medium 12 transmits the forces applied by the medical professional in a manner that more evenly distributes the pressure applied to a radiation shield 14.

Flexible medium 12 is operatively engaged to radiation shield 14. Such operational engagement can be made by affixing flexible medium 12 and radiation shield 14 to one another by a variety of manners well known in the art, including, but not limited to, adhesives, connectors, stitching, and the like.

Radiation shield 14 can be formed from any one of a number of materials known in the art that reduce the number of radioactive particles emanating from isotope seeds 20 from being transmitted to the medical professional. Such materials can include, but are not limited to, lead, polymers (including Plexiglas®), and the like.

Radiation shield 12 additionally serves to transmit the forces resulting from the application of pressure by the medical professional to a patient 22. As a result of the surface area of radiation shield 14, the forces applied by the medical professional are more evenly distributed to the patient. Additionally, radiation shield 14 may be formed in a variety of shapes so as to be well suited for numerous body regions of the patient. Thus, radiation shield 14 may be planar, curved, or in a likewise configuration.

The pressure applied to the perineum stops the bleeding and precludes or reduces edema in the area of implantation and surrounding stressed tissue. In the preferred embodiment, as shown in FIG. 1, a flexible medium is placed in an abutting or engaging relationship to a shielding material, such as lead or Plexiglas®. As force is applied to the flexible medium, the flexible medium transfers and distributes the force more evenly to the shielding material for a more even distribution of pressure to the patient. The more even distribution of force provides a better method of reducing or eliminating edema or bleeding in the patient.

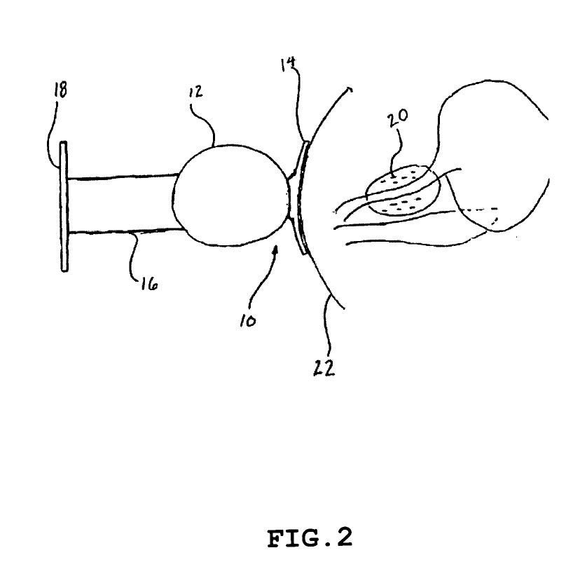

Referring now to FIG. 2, an alternative embodiment of the present invention is shown incorporating a pressure applicator 16. Pressure applicator 16 is operatively engaged to flexible medium 12 in a manner as described above for the operative engagement of flexible medium 12 to radiation shield 14. Pressure applicator 16 is used as a further way in which pressure can be applied by the medical professional to flexible medium 12. Pressure applicator 16 can further include a brace 18. Brace 18 permits the medical professional to transmit force to flexible medium 12 by leaning against pressure placement device 10.

Although only a few exemplary embodiments of the present invention have been described in detail above, those skilled in the art will readily appreciate that numerous modifications are to the exemplary embodiments are possible without materially departing from the novel teachings and advantages of this invention. Accordingly, all such modifications are intended to be included within the scope of this invention as defined in the following appended claims.

What is claimed is:

1. A shielded pressure placement device for the reduction of bleeding or edema and exposure to radiation, comprising:
a flexible medium for receiving an application of pressure;
a radiation shield operatively engaged to said flexible medium; and
wherein said radiation shield transmits said application of pressure to a patient to reduce bleeding or edema and wherein said radiation shield reduces radiation exposure to a user.

2. The shielded pressure placement device of claim 1 wherein said radiation shield is affixed to said flexible medium.

3. The shielded pressure placement device of claim 1 further comprising a pressure applicator.

4. The shielded pressure placement device of claim 3 wherein said pressure applicator further includes a brace.

5. The shielded pressure placement device of claim 3 wherein said pressure applicator is operationally engaged to said flexible medium.

6. The shielded pressure placement device of claim 1 wherein said flexible material is selected from the group consisting of rubbers, polymers, gels, and foams.

7. The shielded pressure placement device of claim 1 wherein said radiation shield is planar.

9. The shielded pressure placement device of claim 1 wherein said radiation shield is curved.

10. A method for applying pressure to a surgical patient, said method comprising the steps of:
applying pressure to a flexible medium, said flexible medium transmitting said pressure to a radiation shield;
transmitting said pressure by said radiation shield to a body region of a surgical patient; and
reducing radiation exposure to a user by said radiation shield.
